(19) United States
(12) Patent Application Publication
(10) Pub. No.: US 2019/0265150 A1
(43) Pub. Date: Aug. 29, 2019

(54) SLOTTED DISCS REPRESENTATIVE OF FORMATION FRACTURES

(71) Applicants: Ian Lee Everhard, Houston, TX (US); Shay Hetz, Florence, SC (US); Grant Mackenzie, Houston, TX (US); Luke Alan Boyer, Houston, TX (US); Reza Ettehadi Osgouei, Spring, TX (US)

(72) Inventors: Ian Lee Everhard, Houston, TX (US); Shay Hetz, Florence, SC (US); Grant Mackenzie, Houston, TX (US); Luke Alan Boyer, Houston, TX (US); Reza Ettehadi Osgouei, Spring, TX (US)

(73) Assignee: Baker Hughes, a GE company, LLC, Houston, TX (US)

(21) Appl. No.: 15/903,753

(22) Filed: Feb. 23, 2018

(51) Int. Cl. G01N 15/08; E21B 21/00; E21B 21/06; G01N 33/28

(52) U.S. Cl. CPC G01N 15/0806; G01N 33/2823; E21B 21/062; E21B 21/003

(57) ABSTRACT

A permeability plugging apparatus disk including a body, and a slot through the body, the slot being non-straight. Also disclosed is method for producing a particulate plugging apparatus disk including estimating a feature to be replicated for particle plugging testing, depositing layer by layer of a material according to a program in an additive manufacturing tool, leaving selective voids in the layer by layer construction to form the disk having a non-straight slot in a body and a permeability plugging apparatus disk including a body defining a pulser screen.

### BACKGROUND

In resource recovery industries, fluid loss control is important. Fluid can be lost to the formation through the fractures and cavities therein that both can harm the formation and be costly in drilling fluids. To alleviate or prevent such losses, lost circulation material is added to drilling fluids. Therefore, it is useful to have particle plugging information to determine what particles should be added to a drilling fluid to help mitigate formation damage by stopping or slowing filtrate invasion into a wellbore fracture or cavity. Traditionally, a Permeability Plugging Apparatus commercially available from OFI Testing Equipment and others is employed to approximate wellbore fractures and cavities to evaluate various lost circulation material constituents for the drilling fluid. To effect the approximation, stainless steel disks with machined slots in them are selected for insertion in the apparatus. A disk 10 of the prior art is illustrated in FIG. 1. It will be appreciated that the slot 12 is simply diametric to the disk and extends through the thickness of the disk in a straight manner and is of smooth interior surface 14 condition. The disk 10 is illustrated in a typical permeability plugging apparatus cell 16 having a housing 18 center piston 20, end cap 22, and a hydraulic pump 24. While these work well for their intended use, the art is always eager for more and better information to help decision making for many varied borehole operations.

### SUMMARY

Disclosed is a permeability plugging apparatus disk including a body, and a slot through the body, the slot being non-straight.

Also disclosed is a method for producing a particulate plugging apparatus disk including estimating a feature to be replicated for particle plugging testing, depositing layer by layer of a material according to a program in an additive manufacturing tool, leaving selective voids in the layer by layer construction to form the disk having a non-straight slot in a body.

Also disclosed is a permeability plugging apparatus disk including a body defining a pulser screen.

### BRIEF DESCRIPTION OF THE DRAWINGS

The following descriptions should not be considered limiting in any way. With reference to the accompanying drawings, like elements are numbered alike:

FIG. 1 is a view of a prior art permeability plugging apparatus disk in a permeability plugging apparatus cell;

FIG. 2 is a view of an example of a permeability plugging apparatus disk as taught herein having a zig-zag slot;

FIG. 3 is a view of an example of a permeability plugging apparatus disk as taught herein having a multi-fingered slot;

FIG. 4A is a view of an example of a permeability plugging apparatus disk as taught herein having a straight slot but with an internal tortuous path visible in FIG. 4B;

FIG. 4B is a cross section view of the disk illustrated in FIG. 4A; and

FIG. 5A is another cross sectional view with a tortuous path but having varying dimensions along its length;

FIG. 5B is an enlarged view of an enlarged view of an inner surface of the permeability plugging apparatus disk shown in FIG. 5A but can be any of the foregoing figures;

FIG. 5C is a more enlarged view of one possible texture of the inner surface shown in FIG. 5B;

FIG. 6 illustrates a disk as taught herein with a screen pattern emulated thereon; and

FIGS. 7A through 7C are varying views of a separable disk as disclosed herein.

### DETAILED DESCRIPTION

A detailed description of one or more embodiments of the disclosed apparatus and method are presented herein by way of exemplification and not limitation with reference to the Figures.

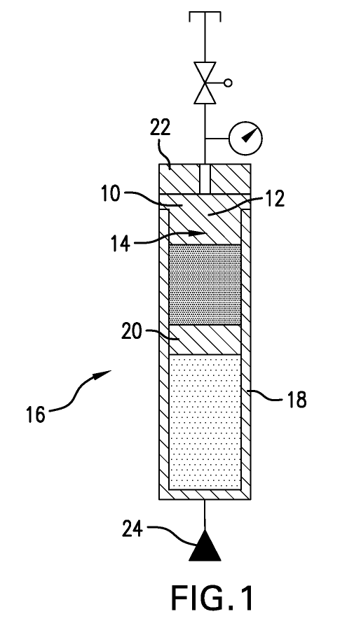

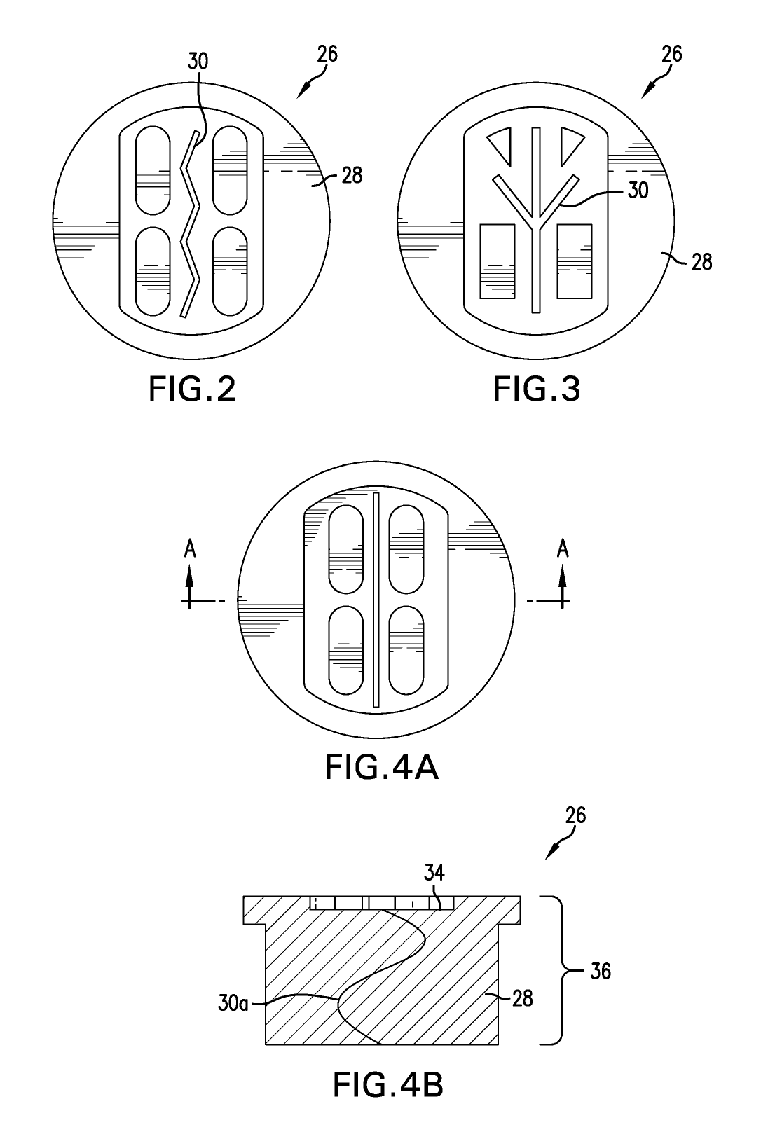

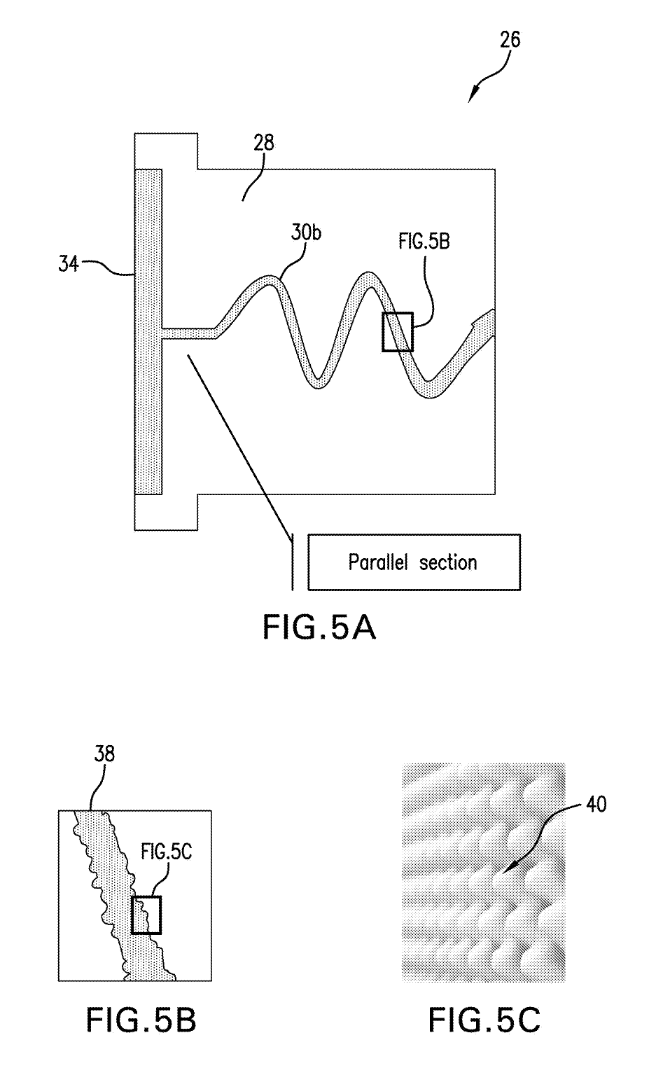

Referring to FIG. 2-5C, a permeability plugging apparatus disk 26 is illustrated. The disk defines a body 28 that may actually be cylindrical or may be provided in other geometric patterns such as square, triangular, rectangular, polygonal, etc. as desired. It will be appreciated immediately that a slot 30 of disk 26 is markedly distinct from the slot 12 of disk 10. Specifically, it will be appreciated that the slot 30 is not straight. Rather the slot 30 may be configured to mimic whatever features of fracture are expected (i.e. zig-zag (FIG. 2), multi-fingered (FIG. 3), variable dimension (FIG. 5A), etc.) in the rock formation that is relevant to the particular test. The slot 30 may have a zig-zag pattern (as shown in FIG. 2) in a contact face 34 of the body 28 and may also have a tortuosity 30a through a thickness 36 of the body 28 (visible in FIGS. 4B and 5A). As noted other features such as cracks with different degrees of opening dimensions (see FIG. 5A numeral 30b for a slot with varying dimensions over its length, for example) or different types of dimension through a thickness 36 are all contemplated. In addition, disks 26 may be provided with texture 40 on an inner surface 38 (see FIGS. 5A-5C) of the slot 30 to adjust flow characteristics therethrough. Textures contemplated include but are not limited to rough consistent patterning. Inconsistent patterning, protrusions numbering one to many, etc. These enhancements to disks for a permeability plugging apparatus are taught herein because the inventors hereof have determined that the industry paradigm, represented in FIG. 1, is not, in fact, sufficient to provide the guidance actually needed for determining what lost circulation material should be added to drilling fluids. More specifically, while the industry has believed that the traditional slotted disk method for testing leads to an accurate representation, what it actually leads to is a larger particulate size and density than is actually necessary, thereby increasing costs. Employing the disks as disclosed herein, therefore has the effect of increasing accuracy and reducing costs for materials.

It is to be understood that all of the above noted features may be employed together in a single disk as illustrated in FIG. 2 or any of the features may be employed alone in a single disk 26. Further, any of the disclosed features may be combined with any one or more of the other of the disclosed features or may be combined with features not disclosed as well and still be within the scope of the invention.

The features noted above would be extremely difficult or impossible to manufacture by traditional subtractive manufacturing or would be tedious and costly to make by manual addition and fusing of components but are easy and economical to produce through the use of additive manufacturing wherein the disks 26 may be built up in a layer-by-layer manner using Direct Metal Laser Sintering, Direct Metal Laser Melting, etc. following a computer modeling program. A computer model of exactly what features are needed may be created using the same estimates presently used for straight slot disks but with significantly more accurate testing results due to the nonstraight, tortuous and surface feature changes according to the teaching hereof. Specifically, traditional estimation of formation features such as fractures and cavities, etc. may be used to develop the computer model used for additive manufacture of disks. Alternatively, it is also contemplated to use texture analysis as a substitute for or in addition to the traditional estimation methods as a basis for at least a portion of the building of the computer program to additively manufacture a disk or a set of disks. Texture analysis is useful for obtaining surface roughness for a given rock surface including frequency of smaller fractures of the rock surface which will add to the roughness of the surface. This information then is added to the program in order to cause a disk created thereby to have features of the disk 26 including the inner surface 38 emulate the frictional factors imposed by the inner walls of a natural fracture in the formation. Texture analysis, as is known to those of skill in the art, refers to the characterization of regions in an image by their texture content and is useful in this context to quantify intuitive qualities described by terms such as rough, smooth, silky, or bumpy as a function of the spatial variation in pixel intensities thereby allowing for representation of such properties in the AM program.

Commercially available software such as ZYGO Mx™ exists that filters an image using standard statistical measures. These statistics can characterize the texture of an image because they provide information about the local variability of the intensity values of pixels in an image. For example, in areas with smooth texture, the range of values in the neighborhood around a pixel is a small value; in areas of rough texture, the range is larger. Similarly, calculating the standard deviation of pixels in a neighborhood can indicate the degree of variability of pixel values in that region.

For the purposes of creating slotted disks as taught herein, a photograph of a fractured rock that is known to be of the type in the formation being emulated, is photographed and interrogated with one of these commercially available programs to arrive at a standard deviation of pixels and therefor a characterization of the rock surface roughness. This information is then used in the AM program to ensure the inner surface 38 is a close representation of the actual formation surfaces.

The disk 26 produced in the manner described allows for better mimicking of the actual fluid dynamic realities in the borehole and therefore better determination of particulate to add to the drilling fluid.

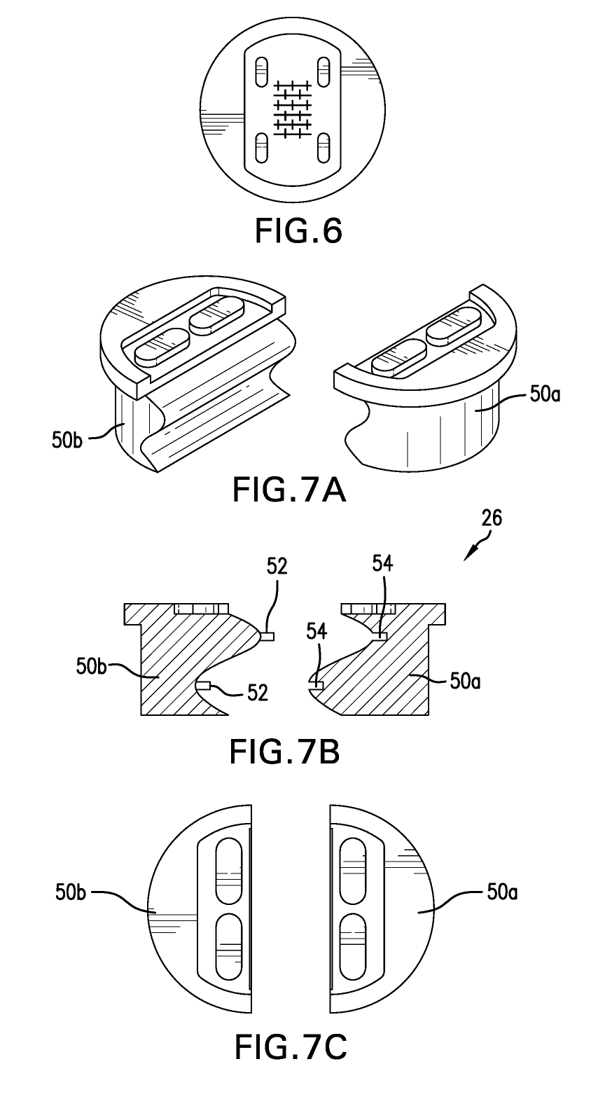

In addition to the foregoing features, and referring to FIG. 6, it is also contemplated that the disk 26 may be configured in a facsimile of a pulser screen in order to utilize the particulate plugging apparatus to determine the likelihood of particulate matter in the drilling fluid plugging a pulser used in the borehole. This utility is somewhat opposite the foregoing utilities in that the use of the particulate plugging apparatus above is to determine which particulates will be sufficient to plug fractures and fishers in the formation to prevent fluid loss thereto while the latter utility is to ensure that particulates selected do not bridge the pulser screens and make pulser communication impossible.

Referring to FIGS. 7A through 7C (which are very similar to the embodiment of FIGS. 4A and 4B), another feature of a disk 50 in portions 50a and 50b is illustrated. It is to be appreciated that this feature may be used with any other feature disclosed above. Disk 50 is separable along its slot. This facilitates both an inspection function such that how the path is plugged with particles may be directly reviewed and allows for easy cleaning of the disk for reuse. In embodiments, the disk 50 may have one or more pins 52 and one or more recesses 54 to align and/or secure the portions of the disk 50. The pins may be a part of the portions 50a or 50b or may exist in the form of fasteners extending through one portion and threadedly connected to the opposite portion.

Set forth below are some embodiments of the foregoing disclosure:

### Embodiment 1

A permeability plugging apparatus disk including a body, and a slot through the body, the slot being non-straight.

### Embodiment 2

The disk as in any prior embodiment wherein the body is cylindrical.

### Embodiment 3

The disk as in any prior embodiment wherein the slot is multifingered.

### Embodiment 4

The disk a as in any prior embodiment wherein the slot is zig-zag shaped.

### Embodiment 5

The disk as in any prior embodiment wherein the slot is of varying dimensions over a length of the slot.

### Embodiment 6

The disk as in any prior embodiment wherein the slot has dimensions that converge through a thickness of the body.

### Embodiment 7

The disk as in any prior embodiment wherein the slot defines an inner surface.

### Embodiment 8

The disk as in any prior embodiment wherein the inner surface is textured.

### Embodiment 9

The disk as in any prior embodiment wherein the slot is of a tortuous form through a thickness of the body.

### Embodiment 10

The disk as in any prior embodiment wherein the body is additively manufactured.

### Embodiment 11

The disk as in any prior embodiment wherein the body is separable along the slot.

### Embodiment 12

A method for producing a particulate plugging apparatus disk including estimating a feature to be replicated for particle plugging testing, depositing layer by layer of a material according to a program in an additive manufacturing tool, leaving selective voids in the layer by layer construction to form the disk having a non-straight slot in a body.

### Embodiment 13

The method as in any prior embodiment further including texturing an inner surface of the slot.

### Embodiment 14

A permeability plugging apparatus disk including a body defining a pulser screen.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The teachings of the present disclosure may be used in a variety of well operations. These operations may involve using one or more treatment agents to treat a formation, the fluids resident in a formation, a wellbore, and/or equipment in the wellbore, such as production tubing. The treatment agents may be in the form of liquids, gases, solids, semi-solids, and mixtures thereof. Illustrative treatment agents include, but are not limited to, fracturing fluids, acids, steam, water, brine, anti-corrosion agents, cement, permeability modifiers, drilling muds, emulsifiers, demulsifiers, tracers, flow improvers etc. Illustrative well operations include, but are not limited to, hydraulic fracturing, stimulation, tracer injection, cleaning, acidizing, steam injection, water flooding, cementing, etc.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A permeability plugging apparatus disk comprising:
a body; and
a slot through the body, the slot being non-straight.

2. The disk as claimed in claim 1 wherein the body is cylindrical.

3. The disk as claimed in claim 1 wherein the slot is multifingered.

4. The disk as claimed in claim 1 wherein the slot is zig-zag shaped.

5. The disk as claimed in claim 1 wherein the slot is of varying dimensions over a length of the slot.

6. The disk as claimed in claim 1 wherein the slot has dimensions that converge through a thickness of the body.

7. The disk as claimed in claim 1 wherein the slot defines an inner surface.

8. The disk as claimed in claim 7 wherein the inner surface is textured.

9. The disk as claimed in claim 1 wherein the slot is of a tortuous form through a thickness of the body.

10. The disk as claimed in claim 1 wherein the body is additively manufactured.

11. The disk as claimed in claim 1 wherein the body is separable along the slot.

12. A method for producing a particulate plugging apparatus disk comprising:
estimating a feature to be replicated for particle plugging testing;
depositing layer by layer of a material according to a program in an additive manufacturing tool;
leaving selective voids in the layer by layer construction to form
the disk having a non-straight slot in a body.

13. The method as claimed in claim 12 further comprising:
texturing an inner surface of the slot.

14. A permeability plugging apparatus disk comprising:
a body defining a pulser screen.
